(19) United States
(12) Patent Application Publication
(10) Pub. No.: US 2005/0055078 A1
(43) Pub. Date: Mar. 10, 2005

(54) Stent with outer slough coating

(75) Inventor: Todd D. Campbell, Petaluma, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/897,042

(22) Filed: Jul. 22, 2004

Related U.S. Application Data
(60) Provisional application No. 60/500,583, filed on Sep. 4, 2003.

(51) Int. Cl. A61F 2/06

(52) U.S. Cl. 623/1.11; 623/1.46; 427/2.25

(57) ABSTRACT

The stent with an outer slough coating 125 of the present invention provides a coated stent having a permanent coating 130 disposed on the stent and a slough coating 125 disposed on the permanent coating 130. The permanent coating 130 includes an anti-proliferative agent and the slough coating 125 includes an anti-inflammatory agent. The slough coating 125 erodes shortly after stent implantation to deliver the anti-inflammatory agent, which treats tissue trauma from the angioplasty and the presence of the stent. Once the slough coating 125 has substantially eroded, the permanent coating 130 delivers the anti-proliferative agent long-term to prevent tissue growth on the stent or within the body lumen, and prevent restenosis. The permanent coating 130 can also include an anti-inflammatory agent.

### RELATED APPLICATIONS

This application claims the benefit of U.S. Provisional Patent Application 60/500,583 filed Sep. 4, 2003.

### TECHNICAL FIELD

The technical field of this disclosure is medical implant devices, particularly, a stent having an outer slough coating.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

To prevent restenosis, short flexible cylinders, or stents, constructed of metal or various polymers are implanted within the vessel to maintain lumen size. The stents acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when the stents are deployed. Stents can also be self-expanding, growing to a final diameter when deployed without mechanical assistance from a balloon or like device.

Stents have been used with coatings to deliver drug or other therapy at the site of the stent. The coating can be applied as a liquid containing the drug or other therapeutic agent dispersed in a polymer/solvent mixture. The liquid coating then dries to a solid coating upon the stent. The liquid coating can be applied by dipping or spraying the stent while spinning or shaking the stent to achieve a uniform coating. Combinations of the various application techniques can also be used.

The purpose of the coating is to provide the drug to the tissue adjacent to the stent, such as the interior wall of an artery or vessel. Typically, the coating is applied as one or more layers over the stent wires. Some coatings containing drugs biodegrade over six months or more to deliver the drugs. This may not provide the most effective therapy, however, because the body's reaction after stent implantation varies with time. Immediately after stent implantation, inflammation and thrombosis may occur due to the tissue trauma from the angioplasty and the presence of the stent. While the inflammation normally subsides after a few days, tissue growth may result in restenosis three to six months after stent implantation.

WIPO International Publication No. WO 00/32255 to Kamath et al. discloses an implantable medical device including at least one composite layer of a bioactive agent and a polymer material and at least a barrier layer positioned over the composite layer and being of thickness adequate to provide a controlled release of the bioactive agent, the barrier layer being applied by a lower energy plasma polymerization process optimally to a thickness of 50-2000 Angstroms.

WIPO International Publication No. WO 00/21584 to Barry et al. discloses a medical device wherein at least a portion of the exterior surface of the medical device is provided with a polymer coating incorporating a solution of at least one substantially water-insoluble drug in a volatile organic solvent and the drug diffuses out of the polymer coating when the medical device is positioned within the body.

WIPO International Publication No. WO 98/56312 to Wang et al. discloses a stent formed of a framework provided with a first layer of a biodegradable polymer and a second outer layer of a biodegradable polymer over the first layer, wherein the outer layer is further characterized in that it is a surface erodible polymer.

WIPO International Publication No. WO 00/45744 to Yang et al. discloses a medical device, such as a stent, which includes a first coating including a drug or therapeutic substance and a relatively inelastic second coating impervious to the therapeutic substance, the second coating fracturing during expansion of the medical device to allow elution of the therapeutic substance through fissures formed through the second coating.

U.S. Pat. No. 5,879,697 to Ding et al. discloses a medical device having a drug-releasing coating wherein the coating comprises at least two layers: an outer layer containing at least one drug-ionic surfactant complex overlying a reservoir layer containing a polymer and the drug which is substantially free of an ionic surfactant.

It would be desirable to have a stent having an outer slough coating that would overcome the above disadvantages.

### SUMMARY OF THE INVENTION

One aspect of the present invention provides a stent having an outer slough coating to provide delivery of a particular therapeutic agent when needed.

Another aspect of the present invention provides a stent having an outer slough coating to deliver anti-inflammatory agents immediately after stent implantation.

Another aspect of the present invention provides a stent having an outer slough coating to deliver anti-proliferative agents from a permanent coating over a prolonged period.

Another aspect of the present invention provides a stent having an outer slough coating able to deliver both anti-inflammatory and anti-proliferative agents from a permanent coating over a prolonged period.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 shows a stent delivery system made in accordance with the present invention.

FIGS. 2 & 3 show a stent and a cross section, respectively, of a coated stent made in accordance with the present invention.

FIG. 4 shows a graph of drug release rate versus time for a coated stent made in accordance with the present invention.

FIG. 5 shows a method of manufacturing a coated stent made in accordance with the present invention.

### DETAILED DESCRIPTION OF THE PRESENTLY PREFERRED EMBODIMENT

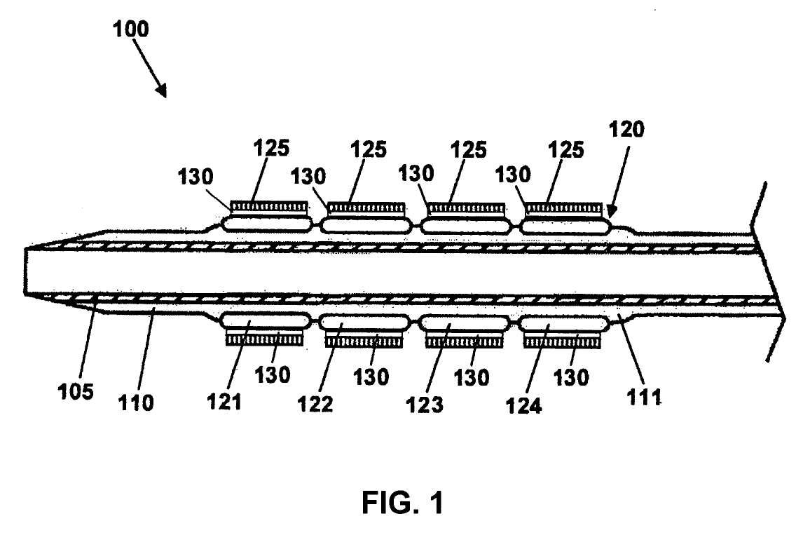

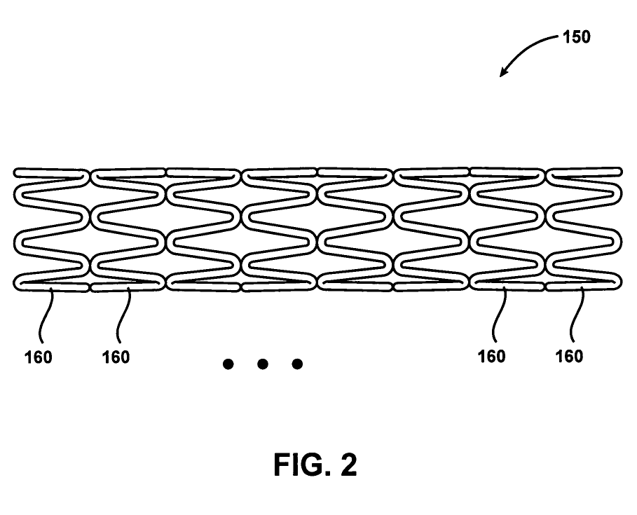

FIG. 1 shows a stent delivery system made in accordance with the present invention. The stent delivery system 100 includes a catheter 105, a balloon 110 operably attached to the catheter 105, and a stent 120 disposed on the balloon 110. The balloon 110, shown in a collapsed state, may be any variety of balloons capable of expanding the stent 120. The balloon 110 may be manufactured from a material such as polyethylene, polyethylene terephthalate (PET), nylon, Pebax® polyether-block co-polyamide polymers, or the like. In one embodiment, the balloon 110 may include retention means 111, such as mechanical or adhesive structures, for retaining the stent 120 until it is deployed. The catheter 105 may be any variety of balloon catheters, such as a PTCA (percutaneous transluminal coronary angioplasty) balloon catheter, capable of supporting a balloon during angioplasty.

The stent 120 may be any variety of implantable prosthetic devices known in the art and capable of carrying a coating. In one embodiment, the stent 120 may have a plurality of identical cylindrical stent segments placed end to end. Four stent segments 121, 122, 123, and 124 are shown, and it will be recognized by those skilled in the art that an alternate number of stent segments may be used. The stent 120 includes at least one slough coating 125 and at least one permanent coating 130. The slough coating 125 is the primary carrier for an anti-inflammatory agent and the permanent coating 130 is the primary carrier for an anti-proliferative agent. The permanent coating 130 can also include additional therapeutic agents, such as an anti-inflammatory agent. In other embodiments, the slough coating 125 and/or the permanent coating 130 can include additional therapeutic agents besides anti-inflammatory agents and anti-proliferative agents. Both the slough coating 125 and the permanent coating 130 can be applied to the stent 120 by dipping or spraying, or a combination of dipping and spraying, as a liquid polymer/solvent mixture containing a drug or other therapeutic agent.

The slough coating 125 and permanent coating 130 are merely exemplary, and it should be recognized that other coating configurations, such as multiple coating layers, are possible. Although the slough coating 125 and the permanent coating 130 are shown schematically on the outer circumference of the stent 120, the slough coating 125 and the permanent coating 130 can coat the whole stent 120, both inside and outside, and around the cross section of individual stent wires.

The slough coating 125 can be any erodible coating that may be eroded from the permanent coating 130 a few days after the stent 120 has been implanted in the patient. The slough coating 125 delivers an anti-inflammatory agent to tissue which may have been injured by angioplasty and stent implantation. The anti-inflammatory agent is delivered immediately after the stent implantation, when it is most useful.

The permanent coating 130 can be any biologically stable, permanent coating that can elute an anti-proliferative agent and maintain coverage of the stent wires. The permanent coating 130 delivers the anti-proliferative agent to prevent tissue growth on the stent or within the body lumen and prevent restenosis.

The anti-proliferative agent is delivered after the slough coating 125 has dissolved, when anti-proliferative agent is most useful. The permanent coating 130 can also include additional therapeutic agents, such as an anti-inflammatory agent.

The slough coating 125 is eroded from the permanent coating 130 to deliver the anti-inflammatory agent, and then the permanent coating 130 elutes the anti-proliferative agent after the slough coating 125 has substantially eroded away. The thickness of the slough coating 125 and the permanent coating 130 can be selected to provide the desired release time for the respective therapeutic agents.

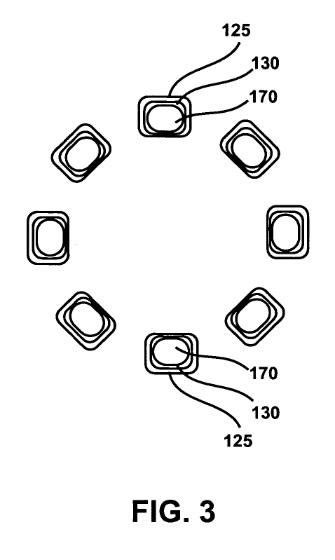

FIG. 2 shows a coated stent made in accordance with the present invention. The stent 150 comprises a number of segments 160. The pattern of the segments 160 can be W-shaped or can be a more complex shape with the elements of one segment continuing into the adjacent segment. The stent 150 can be installed in the stent delivery system of FIG. 1 for implantation in a body lumen.

Referring to FIG. 2, the stent 150 is conventional to stents generally and can be made of a wide variety of medical implantable materials, such as stainless steel (particularly 316-L or 316LS stainless steel), MP35 alloy, nitinol, tantalum, ceramic, nickel, titanium, aluminum, polymeric materials, tantalum, MP35N, titanium ASTM F63-83 Grade 1, niobium, high carat gold K 19-22, and combinations thereof. The stent 150 can be formed through various methods as well. The stent 150 can be welded, laser cut, molded, or consist of filaments or fibers which are wound or braided together in order to form a continuous structure. Depending on the material, the stent can be self-expanding, or be expanded by a balloon or some other device. The slough coating and permanent coating can be disposed on the surface of the segments 160.

FIG. 3 shows a cross section of a coated stent made in accordance with the present invention. A plurality of stent wires or elements 170 are provided with a slough coating 125 and permanent coating 130. The stent wires form the segments which form the stent. Although the cross section of the stent wires or elements 170 is shown as generally rectangular with rounded corners, the cross section can be any number of shapes depending on fabrication methods, materials, and desired effect.

The slough coating 125 is disposed over the permanent coating 130, which is disposed over the stent wires or elements 170. The slough coating 125 includes an anti-inflammatory agent. The permanent coating 130 includes an anti-proliferative agent and, optionally, an anti-inflammatory agent. The coating thicknesses can be selected so that substantially all of the slough coating 125 erodes away and substantially all of the anti-inflammatory agent is released, before substantially any of the anti-proliferative agent is released from the permanent coating 130. Substantially all the slough coating 125 can be considered to have eroded away when less than about 3 to 5 microns, and typically about 2 microns, of the original thickness remains. The slough coating 125 can have a thickness from about 5 to 25 microns, and is typically less than about 15 microns thick. The permanent coating 130 can have a thickness from about 2 to 10 microns, and is typically less than about 5 microns thick. The permanent coating 130 can have a thickness as required to remain intact with the load of the anti-proliferative agent. In one embodiment, the slough coating 125 and the permanent coating 130 can be non-covalently bonded to each other to promote erosion of the slough coating 125. In another embodiment, the slough coating 125 can be thicker than the permanent coating 130.

The slough coating 125 comprises an erodible polymer matrix and an anti-inflammatory agent dispersed in the polymer matrix. The polymer matrix, including one or more polymers, forms the bulk of the slough coating 125. The anti-inflammatory agent can be dissolved throughout the polymer matrix, or can be dispersed throughout the polymer matrix in discrete particles like nano-particles. Nano-particles are typically small particles of crystalline therapeutic agents ground to a small size, such as nanometer-sized particles. Nano-particles can increase the speed of delivery of the anti-inflammatory agent because of the large surface area to volume ratio. In one embodiment, the polymer matrix can contain voids to enhance the erodibility of the slough coating 125. The slough coating 125 can have an anti-inflammatory agent loading from about 10 to 70 weight percent, and is typically greater than about 30 weight percent. The slough coating 125 can have a thickness as required to remain substantially intact with the load of the anti-inflammatory agent as the thickness erodes. In another embodiment, the slough coating 125 can comprise a single drug or multidrug compound and the polymer matrix can be omitted.

In one embodiment, the erodible material for the slough coating 125 can be a natural polymer, such as a carbohydrate or gelatin, or a synthetic polymer, such as glycolide or high co-glycolide polymer. Erodible materials that can be used for the slough coating 125, include, but are not limited to, poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid, hydrogels, polyhydroxyacids, polysaccharides, polyamines, polyaminoacids, polyamides, polycarbonates, silk, keratin, collagen, gelatin, fibrinogen, elastin, actin, myosin, cellulose, amylose, dextran, chitin, glycosaminoglycans, proteins, and combinations, bi-polymers, and co-polymers thereof. In another embodiment, the erodible material for the slough coating 125 can be a nitric oxide-releasing compound. Nitric oxide-releasing polymeric materials are discussed in U.S. Pat. No. 5,994,444 to Trescony et al., assigned to the assignee of the present invention, and incorporated herein by reference.

In one embodiment, the anti-inflammatory agent can be a steroid. Anti-inflammatory agents that can be used in the slough coating 125, include, but are not limited to, steroidal anti-inflammatory agents, non-steroidal anti- inflammatory agents, hydrocortisone, hydrocortisone acetate, dexamethasone, dexamethasone 21-phosphate, fluocinolone, medrysone, prednisolone acetate, fluoromethalone, betamethasone, triaminolone, ibuprofen, ketoprofen, piroxicam, naproxen, sulindac, choline subsalicylate, diflunisal, fenoprofen, indomethacin, meclofenamate, salsalate, tolmetin, magnesium salicylate, diclofenac, enoxaprin, angiopeptin, monoclonal antibodies, hirudin, acetylsalicylic acid, amlodipine, doxazosin or any analogs or any combinations thereof.

The permanent coating 130 comprises a stable polymer matrix and an anti-proliferative agent dispersed in the polymer matrix. The polymer matrix, including one or more polymers, forms the bulk of the permanent coating 130. The anti-inflammatory agent can be dissolved throughout the polymer matrix, or can be dispersed throughout the polymer matrix in discrete units like nano-particles. Nano-particles are typically small particles of crystalline therapeutic agents ground to a small size, such as nanometer-sized particles. Nano-particles can increase the speed of delivery of the anti-proliferative agent because of the large surface area to volume ratio. In one embodiment, the polymer matrix can be free of voids to enhance the stability of the permanent coating 130. The permanent coating 130 can have an anti-proliferative agent loading from about 10 to 90 weight percent, and is typically greater than about 30 weight percent. Typical values for the anti-proliferative agent loading are between about 50 and 70 weight percent. The loading can depend on keeping the polymer relatively intact and not overly depleted after the anti-proliferative agent has been released. In another embodiment, the permanent coating 130 can also include an anti-inflammatory agent. The permanent coating 130 can have an anti-inflammatory agent loading from about 10 to 20 weight percent, and is typically greater than about 15 weight percent. Generally, the combined anti-proliferative and anti-inflammatory drug weight percent is less than about 90 weight percent.

In one embodiment, the stable material of the permanent coating 130 can be made of a phosphorylcholine polymer from Biocompatibles International plc as set forth in U.S. Pat. No. 5,648,442. Other materials for the permanent coating 130 include, but are not limited to, polydioxanone, polyglycolic acid (PGA), polylactic acid (PLA), PGA/PLA copolymers, polycaprolactone, poly epsilon caprolactone, poly-b-hydroxybutyrate (PHB), polyethylene oxide (PEO), polyanhydrides, polyphosphazenes, poly(orthoesters), polyurethane, polysiloxane, and combinations, bi-polymers, and co-polymers thereof. In another embodiment, the stable material of the permanent coating 130 can be a nitric oxide-releasing compound. Nitric oxide-releasing polymeric materials are discussed in U.S. Pat. No. 5,994,444 to Trescony et al., assigned to the assignee of the present invention, and incorporated herein by reference.

In one embodiment, the anti-proliferative agent in the permanent coating 130 can be the drug 42-Epi-(tetrazolyl)-rapamycin, set forth in U.S. Pat. No. 6,329,386 assigned to Abbott Laboratories, Abbott Park, Ill. Other anti-proliferative agents that can be used in the permanent coating 130, include, but are not limited to, ABT-578 tetrazole-containing macrocyclic immunosuppressant from Abbott Laboratories, rapamycin, statins, actinomycin, paclitaxel, 5-fluorouracil, cisplatin, vinblastine, vincristine, epothilones, methotrexate, azathioprine, adriamycin, mutamycin, endostatin, angiostatin, thymidine kinase inhibitors, taxol, and any analogs thereof and any combinations thereof. In another embodiment, the permanent coating 130 can include prohealing compounds.

Anti-inflammatory agents that can optionally be used in the permanent coating 130, include, but are not limited to, steroidal anti-inflammatory agents, non-steroidal anti- inflammatory agents, hydrocortisone, hydrocortisone acetate, dexamethasone, dexamethasone 21-phosphate, fluocinolone, medrysone, prednisolone acetate, fluoromethalone, betamethasone, triaminolone, ibuprofen, ketoprofen, piroxicam, naproxen, sulindac, choline subsalicylate, diflunisal, fenoprofen, indomethacin, meclofenamate, salsalate, tolmetin, magnesium salicylate, diclofenac, enoxaprin, angiopeptin, monoclonal antibodies, hirudin, acetylsalicylic acid, amlodipine, doxazosin and any analogs thereof and any combinations thereof.

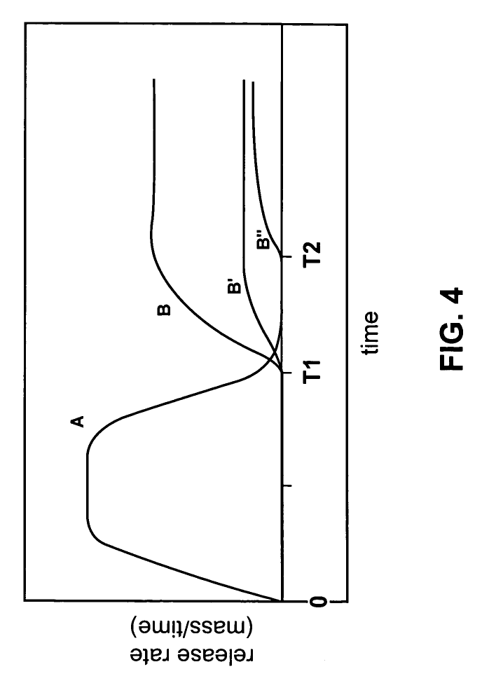

FIG. 4 shows a graph of drug release rate versus time for a coated stent made in accordance with the present invention. The drug release timing can be keyed to the vascular repair mechanism and the onset of smooth muscle cell proliferation to optimize effectiveness.

The coated stent is implanted at time zero and releases an anti-inflammatory agent from the slough coating at the release rate shown in curve A. The anti-inflammatory agent is most effective immediately after stent implantation to treat the tissue trauma from angioplasty and stent implantation. At a predetermined time T1, such as about 14 days, the release rate of the anti-inflammatory agent declines as substantially all of the slough coating erodes away. Substantially all of the slough coating can be considered to have eroded when the less than 2 weight percent of the slough coating remains. At the same predetermined time T1, the release of the anti-proliferative agent from the permanent coating begins, as shown by the release rate in curve B. The anti-proliferative agent is most effective long term after stent implantation to prevent tissue growth on the stent and restenosis. The anti-proliferative agent elutes from the permanent coating for a number of months, generally about 1 to 9 months, and typically about 1-3 months.

The permanent coating can also include an anti-inflammatory agent. The anti-inflammatory agent is effective long term after stent implantation to reduce tissue inflammation and irritation from the stent. In one embodiment, the permanent coating can also include an anti-inflammatory agent that begins to release at the same predetermined time T1 as the anti-proliferative agent, as shown by the release rate in curve B′. In another embodiment, the permanent coating can also include an anti-inflammatory agent that begins to release at a later predetermined time T2 after the anti-proliferative agent, as shown by the release rate in curve B″. The later release can be accomplished by selection and preparation of the anti-inflammatory agent and permanent coating materials. For example, the anti-inflammatory agent can be in the form of coated particles or coated nano-particles embedded within the permanent coating, so that the particle coating degrades before the anti-inflammatory agent was released.

Those skilled in the art will appreciate that FIG. 4 is exemplary only and that the release rate and timing of the different therapeutic agents can be easily varied. Different embodiments can vary parameters such as coating thickness, coating material, and therapeutic agent structure to achieve a desired result.

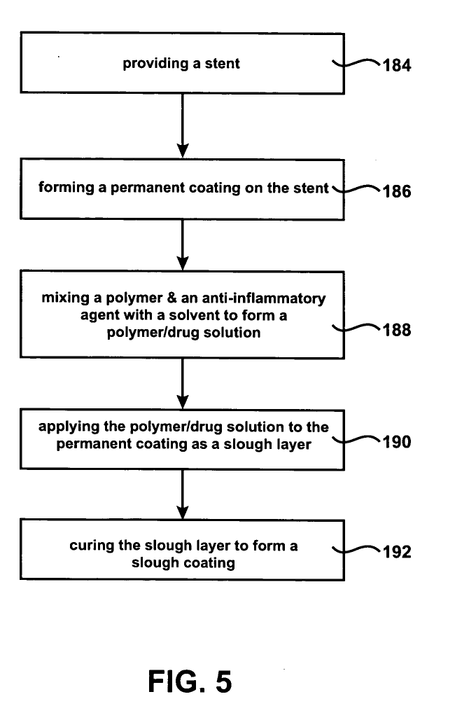

FIG. 5 shows a method of manufacturing a coated stent made in accordance with the present invention. At 184, a stent is provided. A permanent coating is formed on the stent at 186. A polymer and an anti-inflammatory agent are mixed with a solvent to form a polymer/drug solution 188. The polymer/drug solution is applied to the permanent coating in a slough layer 190 and the slough layer cured to form a slough coating 192. The polymer/drug solutions can be applied by spraying, dipping, painting, wiping, rolling, printing, or combinations thereof.

The slough coating can be formed including pores to hasten the erosion of the slough coating. In one embodiment, the polymer/drug solutions can incorporate a gas that forms a foam-like compound and leaves pores. In another embodiment, the polymer/drug solutions can incorporate a sublimating solid, such as dry ice (frozen carbon dioxide), that later evaporates and leaves pores. In another embodiment, the polymer/drug solutions can incorporate a soluble granule, such as a water-soluble salt, that can be washed from the slough coating leaving a pore system.

In one embodiment, forming the permanent coating on the stent comprises mixing a first polymer and an anti-proliferative agent with a first solvent to form a first polymer/drug solution, applying the first polymer/drug solution to the stent as a permanent layer, and curing the permanent layer to form the permanent coating. The first polymer/drug solution can be applied by spraying, dipping, painting, wiping, rolling, printing, or combinations thereof. Optionally, an anti-inflammatory agent can be mixed with the first polymer and the anti-proliferative agent in forming the first polymer/drug solution, so that the permanent coating also includes an anti-inflammatory agent.

In another embodiment, forming the permanent coating on the stent comprises mixing a first polymer with a first solvent to form a polymer solution, applying the polymer solution to the stent as an intermediate layer, curing the intermediate layer to form an intermediate coating, soaking the intermediate coating in a solution including an anti-proliferative agent, and curing the intermediate coating to form the permanent coating. The polymer solution can be applied by spraying, dipping, painting, wiping, rolling, printing, electrostatic deposition, vapor deposition, epitaxial growth, or combinations thereof. Optionally, an anti-inflammatory agent can be mixed with the first polymer in forming the polymer solution, so that the permanent coating also includes an anti-inflammatory agent.

The therapeutic agents in the coatings can take various forms. In one embodiment, the anti-proliferative agent and/or the anti-inflammatory agent can be well dispersed by solubilizing the therapeutic agents in their respective solvents. In another embodiment, the therapeutic agents can be dispersed as discrete particles by maintaining the therapeutic agents as discrete particles in the solvent. The discrete particles can be nano-particles, which increase the speed of delivery of the therapeutic agent because of their large surface area to volume ratio. In addition, the nano-particles leave pores as they dissolve, increasing the erosion rate of the slough coating if desired.

Those skilled in the art will appreciate that the methods of manufacture can be varied for the materials used and the results desired. For example, curing can be omitted or can be a simple drying process for certain polymer and polymer/drug solutions.

It is important to note that FIGS. 1-5 illustrate specific applications and embodiments of the present invention, and is not intended to limit the scope of the present disclosure or claims to that which is presented therein. For example, the slough coating and permanent coating can be applied in a variety of conventional ways, including painting, spraying, dipping, wiping, electrostatic deposition, vapor deposition, epitaxial growth, combinations thereof, and other methods known to those of ordinary skill in the art. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A stent delivery system comprising:
a catheter 105;
a balloon 110 operably attached to the catheter 105; and
a stent 120 disposed on the balloon 110;
a permanent coating 130 disposed on the stent 120, the permanent coating 130 comprising a first polymer matrix and an anti-proliferative agent dispersed in the first polymer matrix; and
a slough coating 125 disposed on the permanent coating 130, the slough coating 125 comprising a second polymer matrix and an anti-inflammatory agent dispersed in the second polymer matrix;
wherein the permanent coating 130 is stable and the slough coating 125 is erodible.

2. The stent delivery system of claim 1 wherein the first polymer matrix is selected from the group consisting of phosphorylcholine, polydioxanone, polyglycolic acid (PGA), polylactic acid (PLA), PGA/PLA copolymers, polycaprolactone, poly epsilon caprolactone, poly-b-hydroxybutyrate (PHB), polyethylene oxide (PEO), polyanhydrides, polyphosphazenes, poly(orthoesters), polyurethane, polysiloxane, nitric oxide-releasing compounds, and combinations, bi-polymers and co-polymers thereof.

3. The stent delivery system of claim 1 wherein the anti-proliferative agent is selected from the group consisting of 42-Epi-(tetrazolyl)-rapamycin, rapamycin, ABT-578 tetrazole-containing macrocyclic immunosuppressant, rapamycin, statins, actinomycin, paclitaxel, 5-fluorouracil, cisplatin, vinblastine, vincristine, epothilones, methotrexate, azathioprine, adriamycin, mutamycin, endostatin, angiostatin, thymidine kinase inhibitors, taxol, any analogs thereof and any combinations thereof.

4. The stent delivery system of claim 1 wherein the permanent coating 130 further comprises a second anti-inflammatory agent dispersed in the first polymer matrix.

5. The stent delivery system of claim 1 wherein the second anti-inflammatory agent is selected from the group consisting of steroids, steroidal anti-inflammatory agents, non-steroidal anti-inflammatory agents, hydrocortisone, hydrocortisone acetate, dexamethasone, dexamethasone 21-phosphate, fluocinolone, medrysone, prednisolone acetate, fluoromethalone, betamethasone, triaminolone, ibuprofen, ketoprofen, piroxicam, naproxen, sulindac, choline subsalicylate, diflunisal, fenoprofen, indomethacin, meclofenamate, salsalate, tolmetin, magnesium salicylate, diclofenac, enoxaprin, angiopeptin, monoclonal antibodies, hirudin, acetylsalicylic acid, amlodipine, doxazosin, any analogs thereof and any combinations thereof

6. The stent delivery system of claim 1 wherein the second polymer matrix is selected from the group consisting of carbohydrates, glycolide, high co-glycolide polymer, poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), poly(ethyleneoxide)-poly(lactic acid), polyalkylene oxalates, polyphosphazenes, biomolecules, fibrin, fibrinogen, starch, collagen, hyaluronic acid, hydrogels, polyhydroxyacids, polysaccharides, polyamines, polyaminoacids, polyamides, polycarbonates, silk, keratin, collagen, gelatin, elastin, actin, myosin, cellulose, amylose, dextran, chitin, glycosaminoglycans, proteins, nitric oxide-releasing compounds, and combinations, bi-polymers and co-polymers thereof.

7. The stent delivery system of claim 1 wherein the anti-inflammatory agent is selected from the group consisting of steroids, steroidal anti-inflammatory agents, non-steroidal anti-inflammatory agents, hydrocortisone, hydrocortisone acetate, dexamethasone, dexamethasone 21-phosphate, fluocinolone, medrysone, prednisolone acetate, fluoromethalone, betamethasone, triaminolone, ibuprofen, ketoprofen, piroxicam, naproxen, sulindac, choline subsalicylate, diflunisal, fenoprofen, indomethacin, meclofenamate, salsalate, tolmetin, magnesium salicylate, diclofenac, enoxaprin, angiopeptin, monoclonal antibodies, hirudin, acetylsalicylic acid, amlodipine, doxazosin, any analogs thereof and any combinations thereof.

8. The coated stent of claim 1 wherein the permanent coating 130 is from about 2 to 15 or 20 microns thick.

9. The coated stent of claim 1 wherein the anti-proliferative agent comprises nano-particles of the anti-proliferative agent.

10. The coated stent of claim 1 wherein the anti-proliferative agent is at least 30 weight percent of the permanent coating 130.

11. The coated stent of claim 1 wherein the slough coating 125 is from about 5 to 25 microns thick.

12. The coated stent of claim 1 wherein the slough coating 125 includes voids.

13. The coated stent of claim 1 wherein the slough coating 125 is of sufficient thickness that substantially all of the slough coating 125 erodes before the permanent coating 130 releases substantially any of the anti-proliferative agent.

14. The coated stent of claim 1 wherein the permanent coating 130 is non-covalently bonded to the slough coating 125.

15. The coated stent of claim 1 wherein the slough coating 125 is thicker than the permanent coating 130.

16. The coated stent of claim 1 wherein the stent comprises a self-expanding stent.

17. A method for producing a coated stent comprising:
providing a stent 184;
forming a permanent coating on the stent, the permanent coating including an anti-proliferative agent 186;
mixing a polymer and an anti-inflammatory agent with a solvent to form a polymer/drug solution 188;
applying the polymer/drug solution to the permanent coating as a slough layer 190; and
curing the slough layer to form a slough coating 192.

18. The method of claim 17 wherein applying the polymer/drug solution to the stent as a slough layer further comprises applying the polymer/drug solution to the permanent coating so as to form pores in the slough layer.

19. The method of claim 17 wherein forming a permanent coating on the stent further comprises:
mixing a second polymer and an anti-proliferative agent with a second solvent to form a second polymer/drug solution;
applying the second polymer/drug solution to the stent as a permanent layer; and
curing the permanent layer to form the permanent coating.

20. The method of claim 17 wherein applying the second polymer/drug solution comprises applying the second polymer/drug solution by an application method selected from the group consisting of spraying, dipping, painting, wiping, rolling, printing, electrostatic deposition, vapor deposition, epitaxial growth, and combinations thereof.

21. The method of claim 19 further comprising mixing a second anti-inflammatory agent with the second polymer/drug solution.

22. The method of claim 17 wherein forming the permanent coating on the stent further comprises:
mixing a second polymer with a second solvent to form a polymer solution;
applying the polymer solution to the stent as an intermediate layer;
curing the intermediate layer to form an intermediate coating;
soaking the intermediate coating in a solution including an anti-proliferative agent; and
curing the intermediate coating to form the permanent coating.

23. The method of claim 22 wherein applying the polymer solution to the stent as an intermediate layer further comprises applying the polymer solution to the stent so as to form voids in the intermediate layer.

24. The method of claim 22 wherein applying the polymer solution to the stent comprises applying the polymer solution by an application method selected from the group consisting of spraying, dipping, painting, wiping, rolling, printing, and combinations thereof.

25. The method of claim 22 further comprising mixing a second anti-inflammatory agent with the polymer solution.
